(19) United States
(12) Patent Application Publication
(10) Pub. No.: US 2019/0046073 A1
(43) Pub. Date: Feb. 14, 2019

(54) PREDICTION APPARATUS AND PREDICTION SYSTEM

(71) Applicant: KYOCERA Corporation, Kyoto (JP)

(72) Inventors: Hirohide YAMASAKI, Yokohama-shi, Kanagawa (JP); Shinichi KOBORI, Saitama-shi, Saitama (JP)

(73) Assignee: KYOCERA Corporation, Kyoto (JP)

(21) Appl. No.: 16/079,753

(22) PCT Filed: Feb. 17, 2017
(86) PCT No.: PCT/JP2017/005974

§ 371 Date: Aug. 24, 2018

(30) Foreign Application Priority Data
Feb. 29, 2016 (JP) 2016-037381

(51) Int. Cl. A61B 5/083; A61B 5/00; A61B 5/08

(52) U.S. Cl. CPC A61B 5/083; A61B 5/7275; A61B 5/082; A61B 5/0803; A61B 5/0022; A61B 5/486

(57) ABSTRACT

A prediction apparatus includes a sensor for detecting a biological gas and a lifelog acquisition unit configured to acquire a lifelog. The prediction apparatus further includes a controller configured to predict a diet effect on the basis of information about the biological gas detected by the sensor and the lifelog acquired by the lifelog acquisition unit.

### CROSS REFERENCE TO RELATED APPLICATION

This application claims priority to and the benefit of Japanese Patent Application No. 2016-037381 filed on Feb. 29, 2016, the entire contents of which are incorporated herein by reference.

### TECHNICAL FIELD

The present disclosure relates to a prediction apparatus and a prediction system for predicting a diet effect.

### BACKGROUND

An activity target calculation method described in PLT 1 set forth below measures an activity level of a user and sets a balance between limitation on meals and fat consumption by exercise for each user. The activity target calculation method described in the PLT 1 sets a moderate diet target tailored to each user.

### CITATION LIST

### Patent Literature

PLT 1: JP-A-2012-165967

### SUMMARY

A prediction apparatus according to an embodiment of the present disclosure includes a sensor for detecting a biological gas and a lifelog acquisition unit configured to acquire a lifelog. The prediction apparatus further includes a controller configured to predict a diet effect on the basis of information about the biological gas detected by the sensor and the lifelog acquired by the lifelog acquisition unit.

The present disclosure may also be implemented as a system substantially equivalent to the prediction apparatus described above. It is appreciated that such a system is also included in the scope of the present disclosure.

A prediction system according to an embodiment of the present disclosure includes a detection apparatus and a prediction apparatus. The detection apparatus includes a sensor for detecting a biological gas, a lifelog acquisition unit configured to acquire a lifelog, and a communication interface for transmitting information about the biological gas detected by the sensor and the lifelog acquired by the lifelog acquisition unit to the prediction apparatus. The prediction apparatus includes a communication interface for receiving the information and the lifelog from the detection apparatus and a controller configured to predict a diet effect on the basis of the information and the lifelog.

A prediction system according to an embodiment of the present disclosure includes a detection apparatus, a relay apparatus, and a prediction apparatus. The detection apparatus includes a sensor for detecting a biological gas, a lifelog acquisition unit configured to acquire a lifelog, and a communication interface for transmitting information about the biological gas detected by the sensor and the lifelog acquired by the lifelog acquisition unit to the relay apparatus. The relay apparatus includes a communication interface for receiving the information about the biological gas and the lifelog from the detection apparatus and transmitting the information about the biological gas and the lifelog to the prediction apparatus via a network. The prediction apparatus includes a communication interface for receiving the information and the lifelog transmitted from the relay apparatus via the network, and a controller configured to predict a diet effect on the basis of the information and the lifelog.

### BRIEF DESCRIPTION OF THE DRAWINGS

In the accompanying drawings:

FIG. 1 is a perspective view schematically illustrating an exterior of a prediction apparatus according to an embodiment of the present disclosure;

FIG. 2 is a functional block diagram schematically illustrating a configuration of the prediction apparatus of FIG. 1;

FIG. 3 is a flowchart illustrating operation of the prediction apparatus of FIG. 1;

FIG. 4 is a functional block diagram schematically illustrating a configuration of a prediction system according to another embodiment of the present disclosure; and

FIG. 5 is a functional block diagram schematically illustrating a configuration of a prediction system according to still another embodiment of the present disclosure.

### DETAILED DESCRIPTION

The activity target calculation method disclosed in the PLT 1 set forth above sets a diet target. This activity target calculation method does not present a detailed future diet effect. A prediction apparatus and a prediction system according to the present disclosure may predict a future diet effect.

Hereinafter, embodiments will be described in detail with reference to the accompanying drawings.

### First Embodiment

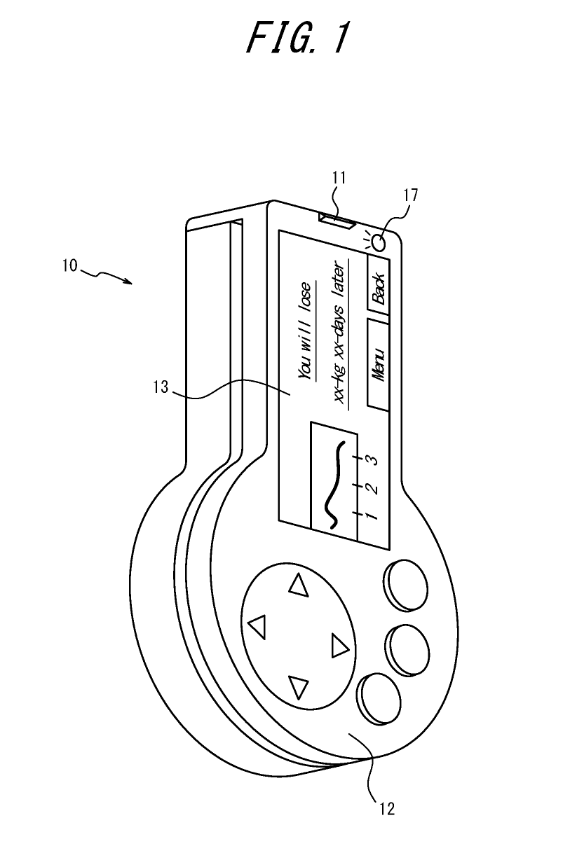

FIG. 1 is a perspective view schematically illustrating an exterior of a prediction apparatus according to a first embodiment. The prediction apparatus 10 includes a sensor 11, an input interface 12, and a display 13.

As illustrated in FIG. 1, the prediction apparatus 10 has a portable clip-on shape. The prediction apparatus 10 is not limited to this shape and may have any portable shape. A user may wear the prediction apparatus 10 by, for example, clipping a clothing pocket.

The prediction apparatus 10 detects a user's state. That is, the prediction apparatus 10 detects movements of a user's body including walking, running, riding a bicycle, driving a car, using an escalator or a lift, moving up/down on a slope, movements during sleep, etc. The user inputs a menu of a meal by using, for example, the input interface 12. The prediction apparatus 10 records a user's lifestyle, i.e., a user's lifelog on the basis of information detected or input. The lifelog is information about a user's movement history including, for example, activities, sleep, and meals.

Based on a recorded lifelog, the prediction apparatus 10 feeds various information back to the user. For example, when the user selects an item associated with a domestic matter or sports, the prediction apparatus 10 calculates the number of steps and calorie consumption and presents them as the lifelog to the user. For example, the prediction apparatus 10 senses user's sleeping hours and movements of the body during sleep, measures the quality of sleep, and presents information about the quality of sleep as the lifelog to the user. For example, the prediction apparatus 10 presents a meal time and calorie intake automatically measured on the basis of the menu input by the user as the life log to the user.

The prediction apparatus 10 may also manage information that greatly affect the lifestyle (lifelog) such as smoking and drinking input by the user. The prediction apparatus 10 may also manage user's physical data including user's height, weight, and blood pressure input by the user. The prediction apparatus 10 may measure an abdominal girth and estimate a visceral fat area.

The prediction apparatus predicts a future diet effect of the user on the basis of the recorded lifelog and information detected by the sensor 11. The prediction apparatus 10 predicts a detailed future diet effect including, for example, weight, a percentage of the visceral fat, and the abdominal girth of the user which the user may lose n-days later, and presents results of the prediction to the user.

As illustrated in FIG. 1, the sensor 11 is arranged on an outer surface of the prediction apparatus 10 in an exposed manner and detects a biological gas discharged from the user. Here, the “biological gas” refers to every gas generated from a living body. That is, the “biological gas” includes various gases included in the exhalation discharged to the outside through the respiratory tract, various gases generated from the skin, and various gases generated from mucous membranes. These biological gases include both organic components and inorganic components. The organic components include, for example, ketones such as acetone, olefins such as isoprene, alcohols such as ethanol, mercaptans such as methyl mercaptan, amine, and ester. The inorganic components include, for example, oxygen, carbon monoxide, carbon dioxide, nitrogen monoxide, ammonia, hydrogen sulfide, and water. The sensor 11 may be implemented by any appropriate sensor capable of detecting the biological gas. For example, a sensor constituting the sensor 11 includes a sensitive film for absorbing gas molecules that constitute the biological gas, and a transducer for converting the gas molecules in the sensitive film into an electrical signal. In order to predict a future diet effect, the sensor 11 needs to include, for example, a sensitive film capable of detecting ketone substances such as acetone.

The sensor 11 may include, for example, a biological gas sensor of a crystal oscillator type including a sensitive film made of an organic thin film and a crystal oscillator. When the gas molecules are absorbed by the sensitive film, a resonant frequency of the crystal oscillator changes, and thus the biological gas sensor of the crystal oscillator type detects the biological gas. The crystal oscillator for detecting the biological gas functions as a transducer for converting the detection of the gas molecules into an electrical signal.

The sensor 11 may include, for example, a semiconductor gas sensor. In the semiconductor gas sensor, when the gas molecules are absorbed by an oxide semiconductor, a resistance value of the oxide semiconductor changes, and thus the semiconductor gas sensor detects gas concentration. The oxide semiconductor functions as a transducer for converting detection of the gas molecules into an electric signal. The sensor 11 may include, for example, an infrared gas sensor, an electrochemical gas sensor, a catalytic combustion gas sensor, or a biosensor.

The input interface 12 is arranged on the exterior of the prediction apparatus 10 in an exposed manner. The input interface 12 receives an input operation from the user. For example, the input interface 12 receives an input operation for selecting an item from the user. For example, the input interface 12 receives an input of a menu of a meal, an input of information (the lifelog) about smoking and drinking, and an input of physical data. The contents input to the input interface 12 by the user are not limited to the above. The input interface 12 may receive any appropriate input that is considered as necessary for implementation of functionality of the prediction apparatus 10. As illustrated in FIG. 1, the input interface 12 includes, for example, operation buttons and operation keys for enabling the user to perform input operations. The input interface 12 may be a touchscreen. The input interface 12 may display an input area for receiving an input operation from the user in a portion of the display 13 and receive a touch operation input from the user.

The display 13 is arranged on the exterior of the prediction apparatus 10 in an exposed manner and adjacent to, for example, the input interface 12. The display 13 displays various items allowing the user to perform input operation, various results measured by the prediction apparatus 10, various data managed by the prediction apparatus 10, and a predicted diet effect of the user. For example, the display 13 displays a selection screen for selecting items such as “activities”, “sleep”, “meal” and “visceral fat” included in “domestic matter” or “sports”. For example, the display 13 displays an input screen for allowing the user to input information about smoking and drinking or physical data. For example, the display 13 displays calculation results of the number of steps and calorie consumption, a measurement result of the quality of sleep, measurement results of a meal time and calorie intake, and an estimation result of the visceral fat area. The display 13 may display the result of each item in a graph statistically indicating daily changes, or in a daily chronological or statistics manner. The display 13 may display a balance between the calorie consumption and calorie intake, e.g., a difference therebetween. The display 13 displays a diet effect that will appear in the user's body or physical condition such as “you will lose n-kg n-days later”, “visceral fat will be reduced by x-%”, “abdominal girth will be reduced by x-centimeters”, “you will wake up feeling refreshed tomorrow morning”, “blood pressure is likely to improve to normal”, etc. Contents displayed on the display 13 are not limited to the above. The display 13 may display any item considered as necessary to implement the functionality of the prediction apparatus 10.

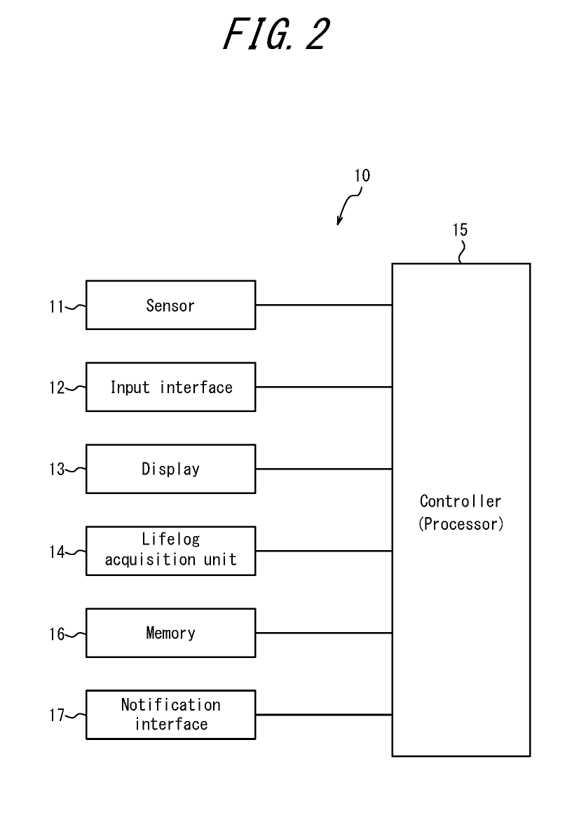

FIG. 2 is a functional block diagram schematically illustrating a configuration of the prediction apparatus 10 of FIG. 1. The prediction apparatus 10 includes a lifelog acquisition unit 14, a controller 15, a memory 16, and a notification interface 17, in addition to the sensor 11, the input interface 12, and the display 13.

The lifelog acquisition unit 14 includes, for example, an acceleration sensor and detects the user's states mentioned above. The lifelog acquisition unit 14 may have any appropriate configuration that is capable of detecting the user's states. The lifelog acquisition unit 14 transmits an acquired lifelog to the controller 15. Based on detected information described above, the controller 15 stores the user's lifelog in the memory 16.

The controller 15 is a processor configured to control and manage the prediction apparatus 10 in its entirety including each functional block thereof. The controller 15 includes a processor such as a CPU (Central Processing Unit) for executing a program specifying a control procedure. Such a program is stored in, for example, the memory 16 or an external storage medium.

As will be described in further detail below, the prediction apparatus 10 includes the controller 15 including at least one processor for perform control and processing for implementing various functions. In various embodiments, the at least one processor may be implemented by an integrated circuit or a plurality of integrated circuits and/or discrete circuits communicably coupled to one another. The at least one processor may be executed according to various known techniques. In one embodiment, the processor includes, for example, one or more circuits or units configured to perform one or more data calculation or data processing by executing a related instruction stored in a memory. In another embodiment, the processor may be a firmware (e.g., a discrete logic component) configured to perform one or more data calculation or data processing. In various embodiments, the processor may include one or more processors, controllers, microprocessors, microcontrollers, integrated circuits for specific application, digital signal processors, programmable logic devices, field programmable gate arrays, any combination of these devices or configurations, or a combination of other known devices or configurations, and implement functions described below.

The controller 15 acquires an input signal on the basis of an operation in respect of the input interface 12 by the user. The controller 15 transmits an output signal to the display 13 as necessary, on the basis of the input signal from the input interface 12. Thus, the controller 15 displays various contents as described above on the display 13.

Data necessary for the display are stored in, for example, the memory 16 or an external storage medium. When the user performs various inputs by operating the input interface 12, the controller 15 acquires data corresponding to the inputs from the memory 16 or the like.

The controller 15 controls the prediction apparatus 10 in its entirety and thus executes prediction processing of the diet effect of the prediction apparatus 10. For example, the controller 15 activates the sensor 11 in response to a predetermined input operation in respect of the prediction apparatus 10 by the user. The predetermined input operation used herein includes, for example, an operation to turn on the power of the prediction apparatus 10 or a selecting operation for executing detection of the biological gas. When activated by the controller 15, the sensor 11 starts detecting the biological gas discharged from the user. The controller 15 acquires information about the biological gas detected by the sensor 11 from the sensor 11.

The controller 15 predicts the diet effect on the basis of, for example, the information about the biological gas detected by the sensor 11 and the lifelog acquired by the lifelog acquisition unit 14. Different biological gases are discharged from the user depending on diet effects. For example, the concentration of acetone generated in the process of lipid metabolism is believed to correspond to an amount of burnt fat. That is, when there is surplus carbohydrate energy in the body, the concentration of acetone decreases. When the carbohydrate energy is insufficient in the body, the concentration of acetone increases. Accordingly, when the user's diet progresses smoothly, the amount of burnt fat gradually increases, thus gradually increasing the concentration of acetone with time. On the other hand, when a progress of diet is disturbed due to rebound or the like, the concentration of acetone rapidly decreases from a high state. Thus, the information about the biometric gas discharged from the user changes in accordance with the progress of the diet. Accordingly, the controller 15 may detects a future diet effect on the basis of a change in the information about the biological gas. Further, the controller 15 acquires the lifelog of each user, and thus performs highly accurate prediction tailored to each user's lifestyle.

For example, the controller 15 may predict the future diet effect on the basis of a ratio of outputs from a plurality of sensors. For example, the controller 15 may detect the future diet effect on the basis of feature quantities (output values, time constants, etc.) of responses from a plurality of sensors. For example, the prediction apparatus 10 may include a plurality of sensors for detecting biological gases including organic components such as ketone, olefin, alcohol, mercaptan, amine, and ester. For example, the prediction apparatus 10 may include a plurality of sensors for detecting biological gases including inorganic components such as oxygen, carbon monoxide, carbon dioxide, nitrogen monoxide, ammonia, hydrogen sulfide, and water. For example, the controller 15 may predict the future diet effect on the basis of a change in outputs of a plurality of sensors for detecting a ketone biological gas.

The controller 15 refers to data indicating a correlation of the information about the biometric gas and the lifelog with the diet effect stored in the memory 16. The controller 15 predicts the diet effect by matching the information about the biometric gas detected by the sensor 11 and the lifelog acquired by the lifelog acquisition unit 14 to the data indicating the correlation. The controller 15 may predict the diet effect by acquiring, as necessary, the information about the detected biological gas, the acquired lifelog, and the data indicating the correlation from responsible units. The controller 15 may predict the diet effect by acquiring the above information at predetermined intervals. The controller 15 may predict the diet effect by acquiring the above information at predetermined intervals set by the user.

The controller 15 stores, in the memory 16, the information about the biological gas acquired from the sensor 11, various information input by the user using the input interface 12, various data necessary for the display by the display 13, and the lifelog acquired from the lifelog acquisition unit 14, as necessary. The controller 15 store a prediction result associated with the future diet effect in the memory 16, as necessary. The controller 15 refers to the data in the memory 16 as necessary.

The controller 15 may predict the diet effect by using a statistical approach such as a principal component analysis or a neural network. The controller 15 may create, in advance, data by practicing learning process for extracting feature values of the responses from the plurality of sensors for each diet effect. The controller 15 may store learned data in the memory 16. The controller 15 may predict the diet effect on the basis of a matching degree between the learned data stored in the memory 16 and the data detected by a plurality of sensors. The controller 15 may update the learned data stored in the memory 16 with data detected anew.

The controller 15 causes the notification interface 17 to notify the user of a prediction result associated with the future diet effect. The controller 15 may notify the user by predicting the future diet effect as necessary. The controller 15 may notify the user by predicting the diet effect at predetermined intervals. The controller 15 may notify the user by predicting the diet effect at predetermined intervals set by the user. The diet effect predicted by the controller 15 includes, for example, a future abdominal girth, a future fat mass, a future physical condition of the user, etc.

The memory 16 may be implemented by a semiconductor memory or a magnetic memory and store various information described above, various data, and programs for operating the prediction apparatus 10. The memory 16 also functions as a working memory. In particular, the memory 16 stores the information about the detected biological gas, the acquired lifelog of the user, and data indicating the correlation of the information about the biological gas and the lifelog with the diet effect

The notification interface 17 notifies the user of the future diet effect predicted by the controller 15. The notification interface 17 may notify, for example, in a visual manner using an image, a character, a color display, or an illuminated display, in an audio manner using a voice or a sound, or a combination thereof. When the notification interface 17 notifies in the visual manner, the notification interface 17 may cooperate with the display 13 or constitute a display device different from the display 13. In this case, the notification interface 17 may notify by displaying an image or a character. The notification interface 17 may notify by, for example, displaying a detailed diet effect such as “you will lose n-kg n-days later” and, simultaneously, illuminating a light emitting element such as an LED. When the notification interface 17 notifies in the audio manner, the notification interface 17 is implemented by a sound generation device such as a speaker and notifies by outputting an alarm sound or a voice guide. The notification by the notification interface 17 is not limited to the visual manner or the audio manner. The notification by the notification interface 17 may be in any manner that enables the user to recognize the detailed diet effect of the user. For example, the notification interface 17 may notify by a vibration in a pattern or the like.

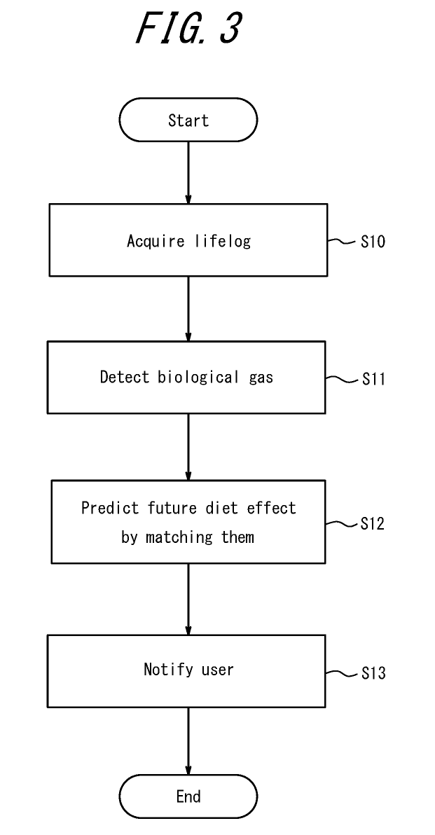

FIG. 3 is a flowchart illustrating operation of the prediction apparatus 10 according to an embodiment.

By using the input interface 12, the user performs a predetermined input operation to start diet effect prediction processing of the prediction apparatus 10. For example, the user operates to select execution of the detection of the biological gas.

After the prediction apparatus 10 starts the prediction processing, the controller 15 acquires the lifelog from the lifelog acquisition unit 14 or the memory 16 (step S10).

The controller 15 activates the sensor 11 to detect the biological gas discharged from the user (step S11). The procedures in steps S10 and S11 are not limited to the above order, and may be executed in a reverse order or simultaneously as parallel procedures.

The controller 15 matches the information about the biological gas detected in step S11 and the lifelog acquired in step S10 to the data indicating the correlation stored in the memory 16 and thus predicts the detailed future diet effect (step S12).

After predicting the diet effect, the controller 15 causes the notification interface 17 to notify the user (step S13). Then, the flow ends.

In this way, the prediction apparatus 10 according to an embodiment may predict the detailed future diet effect.

The prediction apparatus 10 according to an embodiment uses the biological gas detected by the sensor 11 and the lifelog acquired from each user and thus is capable of highly accurately predicting the diet effect tailored to the lifestyle of each user.

The prediction apparatus 10 according to an embodiment presents a detailed diet effect and thus may motivate the user to go on a diet.

The prediction apparatus 10 according to an embodiment has excellent portability and thus may be more convenient for the user.

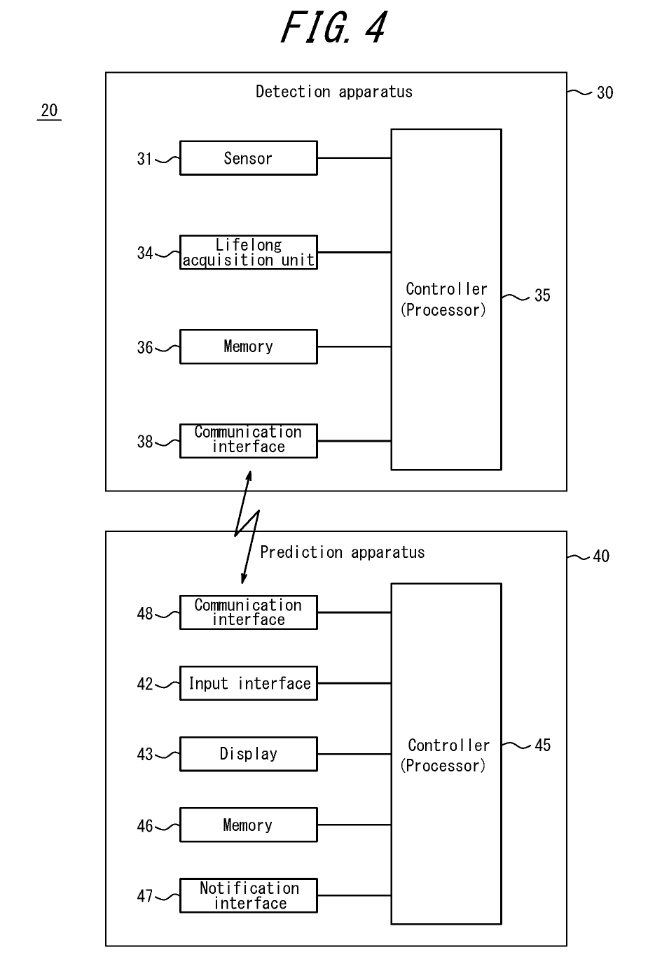

The prediction apparatus 10 according to the above embodiment is described as including the sensor 11 for detecting the biological gas, the lifelog acquisition unit 14 configured to acquire the lifelog, and the controller 15 configured to predict a detailed future diet effect. However, different apparatuses communicable with one another may include a functional unit configured to detect the biological gas, a functional unit configured to acquire the lifelog, and a functional unit configured to predict a detailed future diet effect, respectively. Configurations in such a case will be described as a second embodiment with reference to FIG. 4.

### Second Embodiment

FIG. 4 is a functional block diagram schematically illustrating a configuration of a prediction system 20 according to the second embodiment. The prediction system 20 includes a detection apparatus 30 and a prediction apparatus 40. The detection apparatus 30 and the prediction apparatus 40 are coupled to each other by a short-distance wireless communication such as Bluetooth® (registered trademark in Japan, other countries, or both), ZigBee® (registered trademark in Japan, other countries, or both), or NFC (Near Field Communication).

Unlike the prediction apparatus 10 according to the first embodiment illustrated in FIG. 1, the detection apparatus 30 does not include an input interface, a display, and a notification interface. The detection apparatus 30 has otherwise the same configuration as the prediction apparatus 10 according to the first embodiment. The detection apparatus 30 includes a sensor 31, a lifelog acquisition unit 34, a controller 35, a memory 36, and a communication interface 38. The sensor 31, the lifelog acquisition unit 34, the controller 35, and the memory 36 function in the same manner as the sensor 11, the lifelog acquisition unit 14, the controller 15, and the memory 16, respectively, of the prediction apparatus 10 illustrated in FIG. 2. Thus, detailed descriptions of them will be omitted here. Hereinafter, features different from those of the prediction apparatus 10 according to the first embodiment will be mainly described.

The controller 35 of the detection apparatus 30 according to an embodiment does not predict a detailed future diet effect. Instead, the controller 35 transmits the information about a biological gas detected by the sensor 31 and a lifelog acquired from the lifelog acquisition unit 34 to the prediction apparatus 40 implemented by an external apparatus via the communication interface 38. Also, the controller 35 acquires a necessary signal in accordance with an input operation in respect of the prediction apparatus 40 by the user. For example, the controller 35 acquires a signal for activating the sensor 31 from the prediction apparatus 40.

The communication interface 38 transmits and receives various information by performing the short-distance wireless communication with the prediction apparatus 40.

The communication interface 38 transmits information about the biological gas acquired from the sensor 31 and the lifelog acquired from the lifelog acquisition unit 34 to the prediction apparatus 40. The transmission of various information from the detection apparatus 30 to the prediction apparatus 40 may be performed each time the controller 35 acquires the various information, or when the user performs a predetermined input operation in respect of the prediction apparatus 40.

Also, the communication interface 38 acquires a necessary signal in accordance with a predetermined input operation in respect of the prediction apparatus 40 by the user. For example, the communication interface 38 receives the signal for activating the sensor 31 from the prediction apparatus 40.

The prediction apparatus 40 may be implemented by a mobile terminal apparatus such as a smartphone. The prediction apparatus 40 includes an input interface 42, a display 43, a controller 45, a memory 46, a notification interface 47, and a communication interface 48. The input interface 42, the display 43, the controller 45, the memory 46, and the notification interface 47 function in the same manner as the input interface 12, the display 13, the controller 15, the memory 16, and the notification interface 17, respectively, of the prediction apparatus 10 illustrated in FIG. 2. Hereinafter, features different from those of the prediction apparatus 10 according to the first embodiment will be mainly described.

The input interface 42 and the display 43 may be collectively implemented by a touchscreen of the prediction apparatus 40 implemented by a mobile terminal apparatus such as a smartphone. The input interface 42 displays an input area for receiving an input operation from the user in a portion of the display 43 and thus receives a touch operation input by the user. The input interface 42 may receive an input of a detailed menu of a meal when the user takes a photograph of a meal by using a camera mounted in the prediction apparatus 40 implemented by a mobile terminal apparatus such as a smartphone. The display 43 may display, on the entire touchscreen, various statistical information indicating a result of each item or various information including a diet effect.

The controller 45 is a processor configured to control and manage the prediction apparatus 40 in its entirety including each functional block thereof. The controller 45 may be implemented by a processor such as a CPU (Central Processing Unit) configured to execute a program specifying a control procedure. Such a program is stored in, for example, the memory 46 or an external storage medium.

The controller 45 predicts a detailed future diet effect on the basis of the various information received from the detection apparatus 30 via the communication interface 48. That is, the controller 45 predicts the detailed future diet effect on the basis of the information about the biological gas detected by the sensor 31 and the lifelog acquired by the lifelog acquisition unit 34. The controller 45 refers to the data indicating the correlation of the information about the biological gas and the lifelog with the diet effect stored in the memory 46. The controller 45 predicts the diet effect by matching the information about the detected biological gas and the acquired lifelog to the data indicating the correlation.

The memory 46 may be implemented by a semiconductor memory or a magnetic memory and store various information and a program for operating the prediction apparatus 40. The memory 46 also functions as a working memory. The memory 46 stores the data indicating the correlation of the information about the biological gas and the lifelog with the diet effect.

The communication interface 48 transmits and receives various information by performing the short-distance wireless communication with the detection apparatus 30.

The communication interface 48 receives, from the detection apparatus 30, the information about the biological gas detected by the sensor 31 and the lifelog acquired from the lifelog acquisition unit 34. The various information may be received from the detection apparatus 30 each time the controller acquires corresponding information, or when the user performs a predetermined input operation in respect of the input interface 42.

Also, the communication interface 48 transmits necessary information to the detection apparatus 30 in accordance with a predetermined input operation in respect of the input interface 42 by the user. For example, the communication interface 48 transmits a signal for activating the sensor 31 to the detection apparatus 30.

In this way, the prediction system 20 according to an embodiment is capable of predicting a detailed future diet effect. Accordingly, an effect similar to that of the prediction apparatus 10 according to the first embodiment may be acquired.

The prediction system 20 according to an embodiment may receive an input of a detailed menu of a meal by taking a photograph of the meal using the camera mounted in the prediction apparatus 40 implemented by a mobile terminal apparatus such as a smartphone. Thus, the prediction system 20 according to an embodiment may predict the diet effect more accurately.

The prediction system 20 according to an embodiment may function in a more convenient manner by using the touchscreen, functioning as the input interface 42, of the prediction apparatus 40 implemented by a mobile terminal apparatus such as a smartphone.

The prediction system 20 according to an embodiment is capable of displaying information in a large display by using the touch screen, functioning as the display 43, of the prediction apparatus 40 implemented by a mobile terminal apparatus such as a smartphone, and thus may be more convenient for the user.

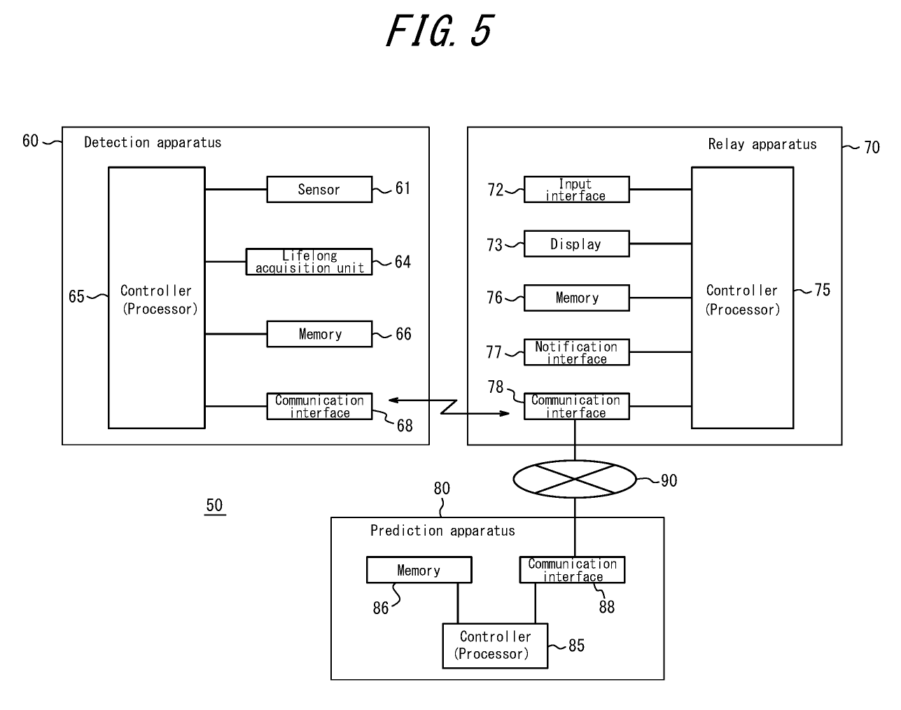

The prediction system 20 according to the above embodiment has been described as a mobile terminal apparatus equipped with a functional unit configured to predict a diet effect for user by the user. However, different apparatuses communicable with one another may include respective functional units configured to predict a diet effect. Configurations in such a case will be described as a third embodiment with reference to FIG. 5.

### Third Embodiment

FIG. 5 is a functional block diagram schematically illustrating a configuration of a prediction system 50 according to the third embodiment. The prediction system 50 includes a detection apparatus 60, a relay apparatus 70, and a prediction apparatus 80. The detection apparatus 60 and the relay apparatus 70 are coupled to each other via the short-distance wireless communication in a manner similar to the second embodiment. The relay apparatus 70 and the prediction apparatus 80 are communicably coupled to each other via a network 90 for a wired or wireless communication such as the Internet, WAN (Wide Area Network), or LAN (Local Area Network).

The detection apparatus 60 and the relay apparatus 70 are configured in a manner similar to the detection apparatus 30 and the prediction apparatus 40, respectively, of the prediction system 20 according to the second embodiment. The detection apparatus 60 includes a sensor 61, a lifelog acquisition unit 64, a controller 65, a memory 66, and a communication interface 68 which function in a manner similar to the sensor 31, the lifelog acquisition unit 34, the controller 35, the memory 36, and the communication interface 38, respectively, of the detection apparatus 30 illustrated in FIG. 4. Thus, detailed descriptions thereof will be omitted here.

The relay apparatus 70 includes an input interface 72, a display 73, a controller 75, a memory 76, a notification interface 77, and a communication interface 78 which function in a manner similar to the input interface 42, the display 43, the controller 45, the memory 46, the notification interface 47, and the communication interface 48, respectively, of the prediction apparatus 40 illustrated in FIG. 4. Thus, detailed descriptions thereof will be omitted here.

Hereinafter, features different from those of the prediction apparatus 10 according to the first embodiment and the prediction system 20 according to the second embodiment will be mainly described.

The controller 75 of the relay apparatus 70 according to an embodiment does not predict a detailed future diet effect. Instead, the controller 75 transmits, via the communication interface 78 and the network 90, information about a biological gas detected by the sensor 61 of the detection apparatus 60 and a lifelog acquired by the lifelog acquisition unit 64 to the prediction apparatus 80 implemented by an external apparatus. Subsequently, the controller 75 acquires, via the communication interface 78, information about a detailed future diet effect transmitted from the prediction apparatus 80 via the network 90. The controller 75 causes the notification interface 77 to notify the user of the information about the diet effect.

The communication interface 78 transmits and receives various information by communicating with the prediction apparatus 80 via the network 90.

For example, the communication interface 78 transmits the information about the biological gas and the lifelog received from the detection apparatus 60 to the prediction apparatus 80. The communication interface 78 also transmits various information input to the input interface 72 to the prediction apparatus 80. The transmission of various information from the relay apparatus 70 to the prediction apparatus 80 may be executed, for example, each time the controller 75 acquires corresponding information, or when the user performs a predetermined input operation in respect of the relay apparatus 70.

Also, the communication interface 78 receives information about the detailed future diet effect predicted by the prediction apparatus 80 from the prediction apparatus 80 via the network 90.

The prediction apparatus 80 may be implemented by, for example, a server apparatus. The prediction apparatus 80 includes a controller 85, a memory 86, and a communication interface 88.

The controller 85 controls and manages the prediction apparatus 80 in its entirety including each functional block thereof. The controller 85 is implemented by a processor such as a CPU (Central Processing Unit) configured to execute a program specifying a control procedure. Such a program is stored in, for example, the memory 86 or an external storage medium.

The controller 85 predicts a detailed future diet effect on the basis of various information received from the relay apparatus 70 via the communication interface 88. That is, the controller 85 predicts the detailed future diet effect on the basis of the information about the biological gas detected by the sensor 61 and the lifelog acquired by the lifelog acquisition unit 64. The controller 85 refers to the data indicating the correlation of the information about the biological gas and the lifelog with the diet effect in the memory 86. The controller 85 predicts the diet effect by matching the information about the biological gas and the lifelog to the data indicating the correlation.

The controller 85 transmits the information about the predicted diet effect to the relay apparatus 70 via the communication interface 88 and the network 90.

The memory 86 may be implemented by a semiconductor memory or a magnetic memory and store various information and a program for operating the prediction apparatus 80. The memory 86 also functions as a working memory. The memory 86 stores data indicating the correlation of the information about the biological gas and the lifelog with the diet effect. The memory 86 stores the information about the biological gas and the lifelog received from the relay apparatus 70. The memory 86 also stores various information input to the input interface 72.

The communication interface 88 transmits and receives various information by communication with the relay apparatus 70 via the network 90.

The communication interface 88 receives the information about the biological gas acquired from the sensor 61 and the lifelog acquired from the lifelog acquisition unit 64 from the relay apparatus 70. The communication interface 88 also receives various information input to the input interface 72 from the relay apparatus 70. The various information may be received from the relay apparatus 70, for example, each time the controller 75 acquires corresponding information, or when the user performs a predetermined input operation in respect of the input interface 72.

The communication interface 88 transmits the information about the detailed future diet effect predicted by the prediction apparatus 80 to the relay apparatus 70 via the network 90.

In this way, the prediction system 50 according to an embodiment may predict a detailed future diet effect. Accordingly, an effect similar to that of the prediction apparatus 10 according to the first embodiment and the prediction system 20 according to the second embodiment may be acquired.

In the prediction system 50 according to an embodiment, the prediction apparatus 80 serving as a server apparatus predicts a detailed future diet effect. Thus, the data stored in the memory 86 and referred to by the controller 85 of the prediction apparatus 80 may be updated as necessary. Thus, the prediction system 50 may predict a diet effect on the basis of updated data.

It is apparent for those who are skilled in the art that the present disclosure may be implemented in manners other than the embodiments described above without departing from the spirit or fundamental features of the present disclosure. Thus, the foregoing description is presented by way of example only and is not restrictive. The scope of the present disclosure is defined by the appended claims, rather than by the foregoing descriptions. In all modifications, modifications within an equivalent scope shall be embraced therein.

For example, a function or the like included in each functional unit or step can be rearranged without a logical inconsistency, such that a plurality of functional units or steps are combined or divided.

For example, in the above embodiment the sensor 11 is implemented by a sensor for detecting the biological gas discharged from the user. However, the sensor 11 may be implemented by another sensor different from the sensor for detecting the biological gas.

Although the prediction system 50 according to the third embodiment is described above as including the detection apparatus 60 and the relay apparatus 70 as separate apparatuses, the detection apparatus 60 and the relay apparatus 70 may be integrally configured as one apparatus.

### REFERENCE SIGNS LIST

10, 40, 80 prediction apparatus
11, 31, 61 sensor
12, 42, 72 input interface
13, 43, 73 display
14, 34, 64 lifelog acquisition unit
15, 35, 45, 65, 75, 85 controller
16, 36, 46, 66, 76, 86 memory
17, 47, 77 notification interface
20, 50 prediction system
30, 60 detection apparatus
38, 48, 68, 78, 88 communication interface
70 relay apparatus
90 network

1. A prediction apparatus comprising:
a sensor for detecting a biological gas;
a lifelog acquisition unit configured to acquire a lifelog; and
a controller configured to predict a diet effect on the basis of information about the biological gas detected by the sensor and the lifelog acquired by the lifelog acquisition unit.

2. The prediction apparatus according to claim 1,
further comprising a memory,
wherein the memory stores data indicating a correlation of the information about the biological gas and the lifelog with the diet effect, and
the controller predicts the diet effect by matching the information about the biological gas and the lifelog to the data.

3. The prediction apparatus according to claim 1,
further comprising a notification interface,
wherein the controller causes the notification interface to notify a predicted diet effect.

4. A prediction system comprising:
a detection apparatus and a prediction apparatus,
wherein the detection apparatus includes a sensor for detecting a biological gas, a lifelog acquisition unit configured to acquire a lifelog, and a communication interface for transmitting information about the biological gas detected by the sensor and the lifelog acquired by the lifelog acquisition unit to the prediction apparatus, and
the prediction apparatus includes a communication interface for receiving the information and the lifelog from the detection apparatus and a controller configured to predict a diet effect on the basis of the information and the lifelog.

5. The prediction system according to claim 4,
wherein the prediction apparatus further includes a memory,
the memory stores data indicating a correlation of the information about the biological gas and the lifelog with the diet effect, and
the controller predicts the diet effect by matching the information about the biological gas and the lifelog to the data.

6. A prediction system comprising:
a detection apparatus, a relay apparatus, and a prediction apparatus,
wherein the detection apparatus includes a sensor for detecting a biological gas, a lifelog acquisition unit configured to acquire a lifelog, and a communication interface for transmitting information about the biological gas detected by the sensor and the lifelog acquired by the lifelog acquisition unit to the relay apparatus,
the relay apparatus includes a communication interface for receiving the information about the biological gas and the lifelog from the detection apparatus and transmitting the information about the biological gas and the lifelog to the prediction apparatus via a network, and
the prediction apparatus includes a communication interface for receiving the information and the lifelog from the relay apparatus via the network, and a controller configured to predict a diet effect on the basis of the information and the lifelog.

7. The prediction system according to claim 6,
wherein the prediction apparatus further includes a memory,
the memory stores data indicating a correlation of the information about the biological gas and the lifelog with the diet effect, and
the controller predicts the diet effect by matching the information about the biological gas and the lifelog to the data.

8. The prediction apparatus according to claim 2,
further comprising a notification interface,
wherein the controller causes the notification interface to notify a predicted diet effect.
